(19) United States
(12) Patent Application Publication
(10) Pub. No.: US 2008/0167685 A1
(43) Pub. Date: Jul. 10, 2008

(54) System and Method For Percutanously Curing An Implantable Device

(75) Inventors: Randy Allard, Germantown, TN (US); Kent M. Anderson, Memphis, TN (US); Aurelien Bruneau, Memphis, TN (US); Eric C. Lange, Collierville, TN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/620,428

(22) Filed: Jan. 5, 2007

(51) Int. Cl. A61B 17/58

(52) U.S. Cl. 606/246

(57) ABSTRACT

A vertebral stabilizing device for stabilizing adjacent vertebrae includes a jacket formed of a biocompatible material and configured for implantation between the vertebrae. The jacket may be configured to encompass a hardenable material. A reaction activator may be encompassed by the jacket.

### FIELD OF THE INVENTION

This disclosure relates to a prosthetic device for supporting and stabilizing the human spine.

### BACKGROUND

Natural spinal discs, extending between adjacent vertebrae in vertebral columns of the human body, provide critical support between the adjacent vertebrae. These discs can rupture, degenerate, and/or protrude by injury, degradation, disease, or the like to such a degree that the intervertebral space between adjacent vertebrae collapses as the disc loses at least a part of its support function. This collapse can cause impingement of the nerve roots and severe pain.

To stabilize and support the spine, and thereby reduce the nerve root impingement and the associated pain, intervertebral prosthetic devices may be implanted between the adjacent vertebrae. These may be implanted both in anterior or posterior areas of the column to prevent the collapse of or maintain the height of the intervertebral space between adjacent vertebrae.

However, different patients often have differently sized spinal columns, differently sized vertebrae, with differently sized intervertebral spaces. Accordingly, a one-size-fits-all approach to intervertebral implantation can be less effective.

Accordingly, what is needed is a vertebral supporting device that can be formed in-situ to provide a desired level of stabilization and support.

### SUMMARY

In one exemplary aspect, this disclosure is directed to a vertebral stabilizing device for stabilizing adjacent vertebrae. The device includes a jacket formed of a biocompatible material and configured for implantation between the vertebrae. The jacket may be configured to encompass a hardenable material. A reaction activator may be encompassed by the jacket.

In another exemplary aspect, this disclosure is directed to a system for stabilizing adjacent vertebrae. The system includes a vertebral stabilizing device having a jacket formed of a biocompatible material and configured for implantation between the vertebrae. The jacket may be configured to encompass a hardenable material. A reaction activator may be encompassed by the jacket. The system also may include a power source configured to power the reaction activator.

In yet another exemplary aspect, a system for posterior stabilization of vertebrae may include a jacket formed of a biocompatible material and being configured for implantation between a first spinous process of an upper first vertebra and a second spinous process of a lower second vertebra to provide posterior support to the first and second vertebrae. A hardenable material may be disposed within the jacket. A reaction activator may be operable to initiate a reaction of the hardenable material to increase the hardness of the hardenable material.

In yet another exemplary aspect, this disclosure is directed to a method of stabilizing adjacent vertebrae. The method may include implanting a jacket formed of a biocompatible material between an upper and a lower vertebra. The jacket may be configured to encompass a hardenable material. The method also may include activating a reaction activator encompassed by the jacket to harden a hardenable material encompassed by the jacket.

In yet another exemplary aspect, this disclosure is directed to a method of stabilizing a posterior portion of vertebrae. The method may include implanting a jacket formed of a biocompatible material between a first spinous process of an upper first vertebra and a second spinous process of a lower second vertebra to provide posterior support to the first and second vertebrae. The jacket may be configured to encompass a hardenable material. The hardenable material may be exposed to a reaction activator source that initiates a reaction of the hardenable material to increase the hardness of the hardenable material.

Various embodiments of the invention may possess one or more of the above features and advantages, or provide one or more solutions to the above problems existing in the prior art.

### BRIEF DESCRIPTION OF THE DRAWINGS

FIG. 1 is a pictorial representation of a side elevation view of an adult human vertebral column.

FIG. 2 is a pictorial representation of a posterior elevation view of the column of FIG. 1.

FIG. 3 is a pictorial representation of an enlarged, top elevation view of one of the vertebrae of the column of FIGS. 1 and 2.

FIG. 4 is a pictorial representation of an enlarged, partial, isometric view of a portion of the column of FIGS. 1 and 2, depicting an exemplary intervertebral prosthetic device inserted between two adjacent vertebrae.

FIG. 5 is a pictorial representation of an enlarged, isometric view of the prosthetic device of FIG. 4.

FIG. 6 is a pictorial representation of another enlarged, isometric view of an exemplary prosthetic device.

FIG. 7 is a pictorial representation of another enlarged, isometric view of an exemplary prosthetic device.

FIG. 8 is a pictorial representation of another enlarged, isometric view of an exemplary prosthetic device.

FIG. 9 is a pictorial representation of another enlarged, isometric view of an exemplary prosthetic device.

FIGS. 10A and 10B are pictorial representations of an implantation procedure of an exemplary prosthetic device between upper and lower vertebrae.

FIGS. 11A and 11B are pictorial representations of an implantation procedure of another exemplary prosthetic device between upper and lower vertebrae.

FIG. 12 is a pictorial representation showing another embodiment of an exemplary implantable device.

FIG. 13 is a pictorial representation showing another embodiment of an exemplary implantable device.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

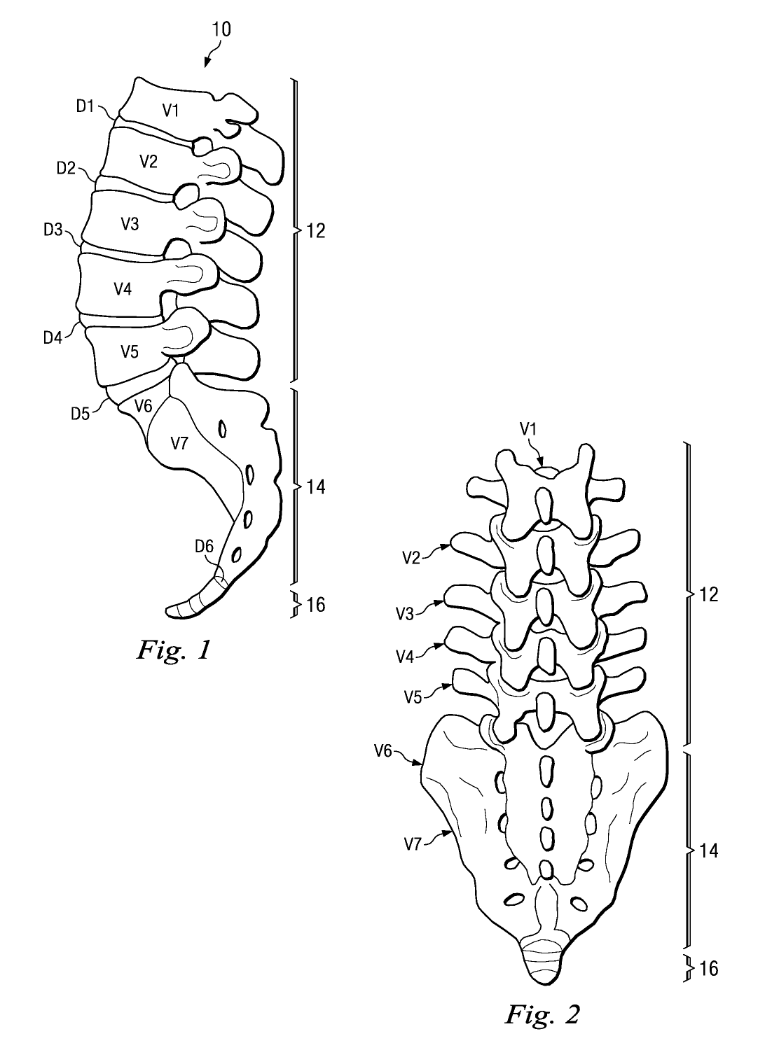

With reference to FIGS. 1 and 2, the reference numeral 10 refers, in general, to a human vertebral column 10. The lower portion of the vertebral column 10 is shown and includes the lumbar region 12, the sacrum 14, and the coccyx 16. The flexible, soft portion of the vertebral column 10, which includes the thoracic region and the cervical region, is not shown.

The lumbar region 12 of the vertebral column 10 includes five vertebrae V1, V2, V3, V4 and V5 separated by intervertebral discs D1, D2, D3, and D4, with the disc D1 extending between the vertebrae V1 and V2, the disc D2 extending between the vertebrae V2 and V3, the disc D3 extending between the vertebrae V3 and V4, and the disc D4 extending between the vertebrae V4 and V5.

The sacrum 14 includes five fused vertebrae, one of which is a superior vertebrae V6 separated from the vertebrae V5 by a disc D5. The other four fused vertebrae of the sacrum 14 are referred to collectively as V7. A disc D6 separates the sacrum 14 from the coccyx 16 which includes four fused vertebrae (not referenced).

With reference to FIG. 3, the vertebrae V4 includes two laminae 20a and 20b extending to either side (as viewed in FIG. 2) of a spinous process 22 that projects posterior from the juncture of the two laminae. Two transverse processes 24a and 24b extend laterally from the laminae 20a and 20b, respectively, and two pedicles 26a and 26b extend inferiorly from the processes 24a and 24b to a vertebral body 28. Since the other vertebrae V1-V3 and V5 are similar to the vertebrae V4 they will not be described in detail.

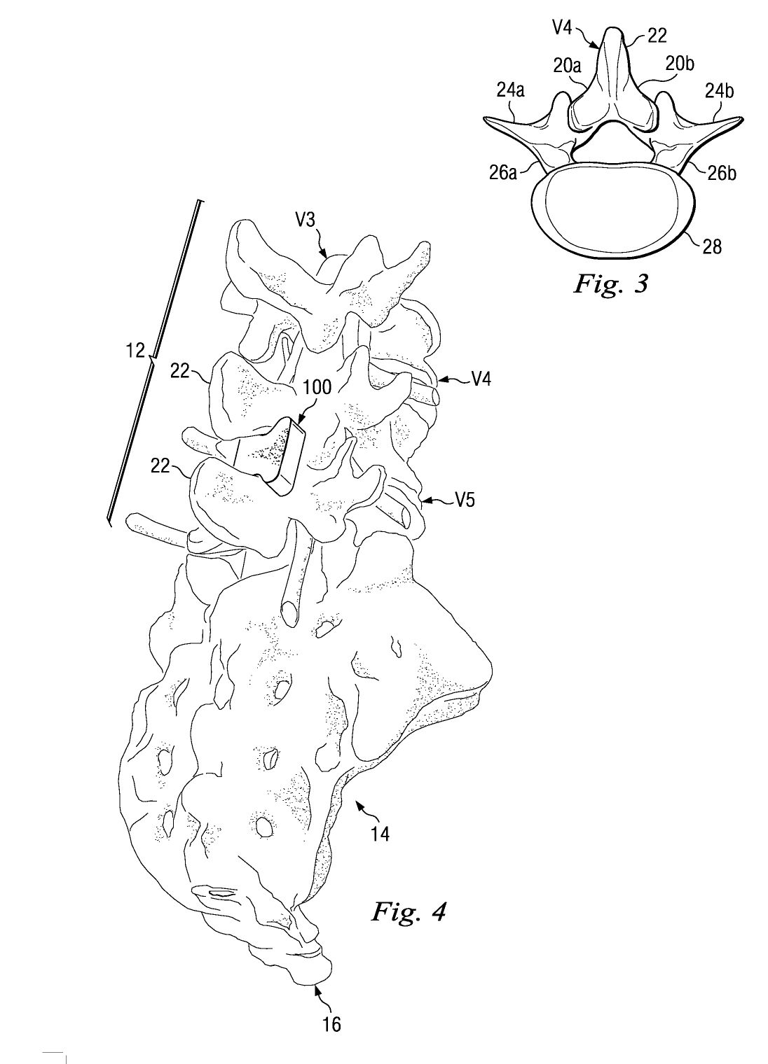

Referring to FIG. 4, it will be assumed that, for one or more of the reasons set forth above, the vertebrae V4 and V5 are not being adequately supported by the disc D4 and that it is therefore necessary to provide supplemental support and stabilization of these vertebrae. To this end, an intervertebral implantable device 100 according to an embodiment of the invention is implanted between the spinous processes 22 of the vertebrae V4 and V5.

The implantable device 100 is configured to be placed between adjacent vertebrae in a formless or amorphous state, and once placed, hardened in-situ to stabilize and support the vertebrae. In some embodiments, the implantable device is configured to be implanted in a deflated state and filled in-situ, while in other embodiments the implantable device 100 is configured to be implanted in a filled state.

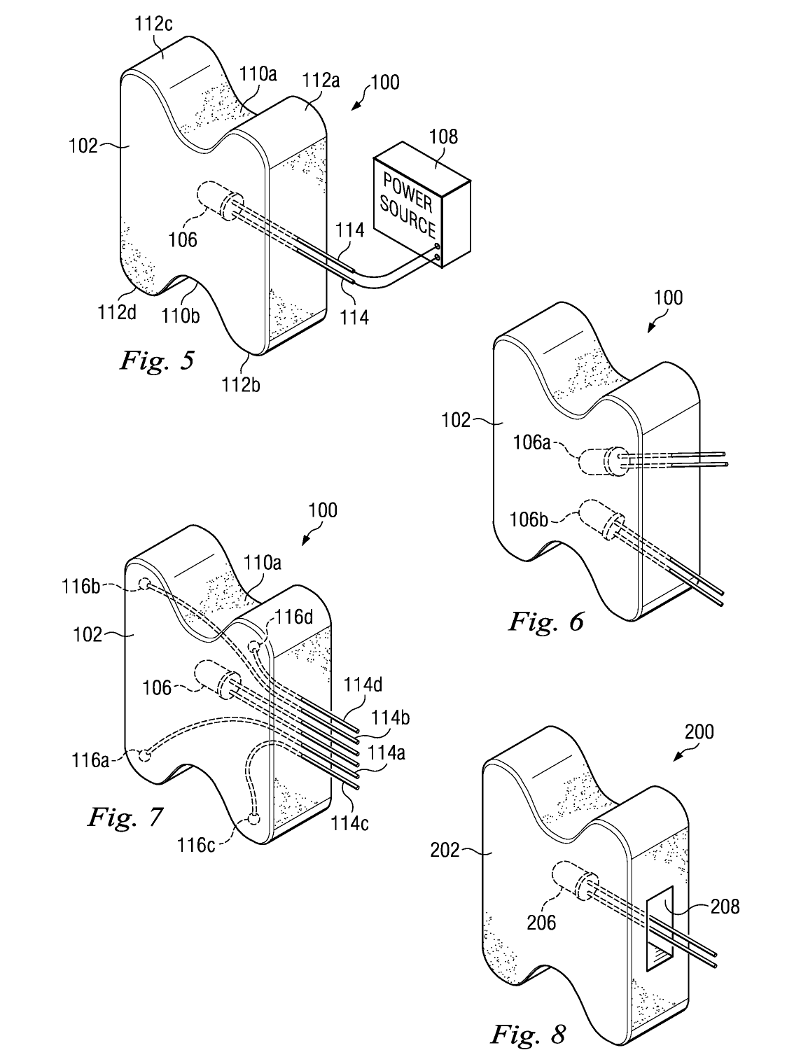

The implantable device 100 is shown in detail in FIG. 5 and is formed in a shape defined by a flexible and deformable jacket 102 with a reaction activator 106 configured to be powered by a power source 108. As described below, the form or shape of the implantable device 100 is defined by a hardenable material encompassed within the jacket 102.

In the embodiment shown in FIG. 5, the jacket 102 is substantially rectangular in shape but includes two curved recesses 110A and 110B formed in its respective end portions. These recesses 110A and 110B separate the end portions into wing portions 112a-d. Referring to FIG. 4, when implanted, the recesses 110A, 110B may receive the spinous processes to distract and cushion the vertebrae. The jacket 102 also may include a port (not shown) that provides access to an interior of the jacket 102. The port may be a hole, a gate, or other feature configured to receive the hardenable material.

In some embodiments, the jacket 102 is formed of an expandable material such as an elastomeric material that may expand and deform. When implanting the jacket in a pre-filled state, this expandable material may aid in manipulating the implantable device into place between spinous processes. In other embodiments the jacket 102 is formed of a substantially non-expandable material, for example, a pliable woven material, that is pre-shaped to take a defined form or shape when filled with the hardenable material. Other embodiments include a combination of expandable and substantially non-expandable materials, allowing the jacket some shape mobility while still providing some pre-defined features. Some of these are described below as shape controllers.

The reaction activator 106 is encompassed by the jacket 102. This allows the reaction activator 106 to initiate a reaction cycle from within the jacket 102 to change the hardenable material from a liquid state, to a tacky, form-holding state, and on to a substantially hardened or hardened state. In the embodiment shown, the reaction activator 106 is a light emitting diode (LED). In some embodiments, the LED is an ultraviolet light emitting diode (UVLED). In yet other embodiments, the reaction activator 106 is an infrared LED (IRLED) and may include a thermistor to regulate the heat source and curing. In yet other embodiments, the reaction activator is a heat generator. Other reaction activators also are contemplated.

In the embodiment shown, the reaction activator 106 is centrally disposed within the jacket 102 and may be configured to provide a radial curing profile extending relatively uniformly towards the jacket. However, other embodiments contemplate locating the reaction activator closer to one edge of the implantable device than another edge to provide a non-uniform curing profile. For example, with reference to FIG. 5, by locating the reaction activator 106 closer to recess 110A than recess 110B, a curing profile may provide unequal, but desired hardening properties at each recess. Likewise, the reaction activator 106 may be disposed at any location within the jacket 106, such as, for example, at ends, in corners, near edges, or at other locations to provide a desired curing profile.

Although in some embodiments the reaction activator is disposed within the jacket 102, in other embodiments, the reaction activator is disposed outside the jacket. In these embodiments, the reaction activator may initiate a reaction that cures the hardenable material from outside the implantable device 100.

In the embodiment shown in FIG. 5, the reaction activator 106 includes leads 114 extending through the jacket wall from the interior of the jacket 102. The leads 114 provide a power connection to activate the reaction activator 106. In other embodiments the reaction activator 106 may be disposed with leads extending through a filling port (not shown). In yet other embodiments, the reaction activator 106 includes no leads extending outside the implantable device 100, but is activated remotely or is self-activated within the implantable device.

Some exemplary hardenable materials include, for example, a single flowable component or may include two or more different flowable components mixed together prior to or during delivery. The hardenable material may further be homogeneous with the same chemical and physical properties throughout, or heterogeneous. A variety of hardenable materials may be used in the present invention and may include polyvinyl chlorides, polyethylenes, styrenic resins, polypropylene, thermoplastic polyesters, thermoplastic elastomers, polycarbonates, acrylonitrile-butadiene-styrene resins, acrylics, polyurethanes, nylons, styrene acrylonitriles, and cellulosics. The hardenable material may further include an opaque additive that will be visible on an X-ray. One type of additive includes barium sulfate.

The power source 108 may be selectively attachable to the leads 114 to power the reaction activator 106. In some embodiments, the power source 108 may be disposed outside a patient's body, while in other embodiments, the power source 108 is disposed within a patient's body, such as adjacent the implantable device 100. In yet other embodiments, the power source 108 is disposed adjacent the reaction activator 106 within the jacket 102. In some embodiments, the power source 108 is external DC power source that may be, for example, battery powered or an active power source. In other embodiments, the power source 108 is a wireless energy source that activates the reaction activator. One example of wireless energy source includes the use electromagnetic waves that generate energy at a coil disposed adjacent the reaction activator 106 to energize the reaction activator. Other power sources 108 also are contemplated. The power source 108 may operate in conjunction with a timer that may apply power for a set period of time (e.g., 30 minutes), may increase or decrease the applied power (e.g. 5-30 Volts) over a set period of time, or otherwise control the power to the reaction activator 106. In some embodiments, an algorithm may be used to determine a desired curing time. Further, curing times and power levels may be based on feedback obtained during the hardening process.

The implantable device 100 also may include more than one reaction activator 106. For example, FIG. 6 shows another embodiment of the implantable device 100 having two reaction activators 106a, 106b disposed within the jacket 102. In the embodiment shown, the reaction activators 106a, 106b are centrally disposed, but in other embodiments, the reaction activators may be disposed at ends, in corners, at edges, at other locations to provide a desired curing profile or to ensure a desired level of curing at these locations. Also, although in FIG. 6 the reaction activators 106a, 106b are shown as being symmetrically disposed, in other embodiments, the reaction activators 106a, 106b are not symmetrically disposed. Varying the location of the reaction activators 106a, 106b can provide a desired curing profile that provides desired properties. It is contemplated that any number of reaction activators may be used to initiate the curing process of the hardenable material. Further, it is contemplated that in some situations, the reaction activators may differ from each other. For example, in one embodiment, the reaction activators may include both a UVLED and an IRLED within the same implantable device 100.

FIG. 7 shows yet another exemplary embodiment of the implantable device 100. Thermocouples 116a-d extend into the jacket 102 to gauge temperatures within the hardenable material during the hardening process. The thermocouples 116a-d include leads 114a-d extending out of the jacket 106. These may connect to a percutaneous meter for determining temperatures detected by the thermocouples 116a-d. In other embodiments, the thermocouples 116a-d are disposed on the exterior of the implantable device 100 or at other locations within the implantable device 100. Although four are shown, any number of thermocouples may be used. In some embodiments, only one is used. In others, two or more are used. Monitoring temperatures may become important when the hardening process is a thermal reaction, to detect when a cure process may be complete or to monitor whether the implantable device 100 is approaching temperatures that may damage living tissue. This may be important when the reaction activator is an IRLED and the hardenable material is cured by heat application.

FIG. 8 shows another embodiment of an implantable device. The implantable device 200 may include any of the features of the implantable device 100 described above, including a jacket 202 and a reaction activator 206. Here however, the implantable device 200 includes a slot 208 configured to receive the reaction activator 206. Accordingly, in this embodiment, the reaction activator 206 may be inserted through the slot 208 within the jacket 202 before, during, or after implantation of the implantable device 200. Therefore, the reaction activator 206 may still initiate hardening of the hardenable material, but also may be removed from the device 200 after hardening. As used herein, a slot is intended to include a slit, a cut, a notch, a recess, an inlet, a port and the like.

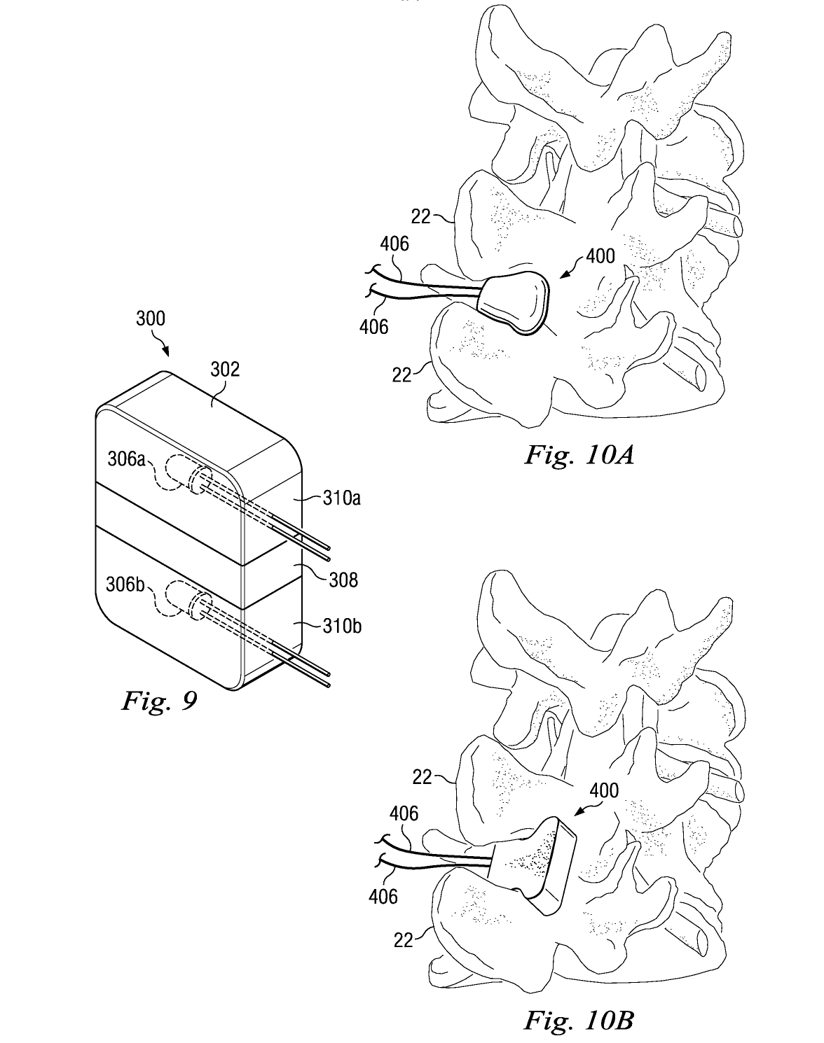

FIG. 9 shows another exemplary embodiment of an implantable device. The implantable device 300 in FIG. 9 is more rectangular-shaped than the embodiments of FIGS. 5-8. Other shapes are contemplated, including for example, square, round, and oval, among others, with any of the shapes having wings, recesses, or other features. The implantable device 300 includes a jacket 302 and two reaction activators 306a, 306b.

The device 300 comprises regions having different properties. Here, the regions include a core 308 and formable ends 310A, 310B. The core 308 may be formed of a solid material that may be not deformable after implantation while the formable ends 310A, 310B may be fillable or filled with hardenable material configured to be hardened by the reaction activators 306a, 306b. Accordingly, curing the hardenable materials may make the device more homogenous, may link the different regions, or provide a gate to a secure position. In other embodiments, one or more of the ends 310a, 310b may be solid material and the core 308 may be fillable or filled with the hardenable material. Naturally, in embodiments having a core of hardenable material, the reaction activator may be disposed adjacent to or in the core. Although shown having three regions, the implantable device 300 may include any number of regions that may be divided along any desired cross-section. For example, in some embodiments, only one wing of the implantable device (such as wing 112a in FIG. 5), only a top portion, or only a bottom portion may be filled with hardenable material and be configured for in-situ hardening. Other divisions or regions are contemplated and would be apparent to one skilled in the art.

Other embodiments may include a passive shape controller, such as a band, that helps control the shape of the implantable device prior to or during the hardening process. For example, referring to FIG. 9, the feature identified by reference numeral 308 may represent a band of material extending about the perimeter of the implantable device 300. This band may have properties that enable it to hold the implantable device in a desired shape or form while the hardenable material solidifies. In some embodiments, the band may be formed of a material having properties rendering it less elastically deformable or less flexible than other regions of the implantable device. Although shown as being around the central portion or waist of the implantable device, such a passive shape controller may be disposed at other locations. For example, it may include multiple bands spaced apart from each other, one or more bands extending perpendicular to that shown, only at the wings 112 a-d (shown in FIG. 5), or in other locations to provide a desired shape. In some embodiments, the passive shape controllers may be at the ends, forming the recesses and wings for receiving bones as in FIG. 5.

In other embodiments, rather than a passive shape controller, any of the implantable devices described may include an active shape controller. These embodiments allow selective shape forming of the implantable devices prior to and during the hardening process. Accordingly, after implantation and prior to or during hardening of the hardenable material, the active shape controller may change or affect the shape or form of the implantable device to render it closer to a desired shape. One example of an active shape controller is a piezoelectric material disposed about or as part of the jacket. Upon inducement of an electrical current or voltage, the material deforms to form the mechanical shape of the implantable device. Upon activation of the piezoelectric material, contraction or expansion of the surface of the implantable device changes the shape to one more desired. While held in that position by the activated piezoelectric material, the reaction activators harden the implantable device in place, or alternatively, the piezoelectric material may alter the mechanical shape during the hardening process. In some embodiments, only a portion of the implantable device is formable using an active shape controller. For example, in some embodiments, upon activation, the active shape controller forms the wings 112a-d and recesses 110a-b of FIG. 5 from what may otherwise be a formless or amorphous shape.

In use, the implantable device may be disposed between spinous processes of a superior and an inferior vertebra. Prior to implantation, the device may contain a flowable or hardenable material, thereby providing some compliable or formless properties, allowing the device to be manipulated into place. Alternatively, a deflated device may be implanted and filled in situ with the hardenable material, through a port (not shown). In these embodiments, the device may expand as it is filled to increase its volume and form in place. No matter when it is filled, the jacket may include a pre-defined shape, may include expandable or non-expandable material, and/or may include shape controllers.

FIGS. 10A and 10B show an implantable device 400 placed to stabilize upper and lower spinous processes 22 according to one embodiment of the present invention. The implantable device 400 may include any of the features of the embodiments described in this disclosure or may be any of the embodiments in this disclosure. In the embodiment of FIG. 10A, the device 400 is pre-filled with hardenable material prior to implantation. Even still, in this embodiment, the device 400 is somewhat amorphous and flowable in its pre-hardened state. Accordingly, it may be placed between and flow to form around misaligned adjacent spinous processes 22. In this embodiment, the reaction activator is disposed within the jacket of the implantable device 400 and therefore is not shown.

Filling the device 400 prior to implantation may eliminate the need for an injecting syringe, ports that must be closed, gates of materials resulting from the ports, and pressure or volume determinations. Accordingly, implantation processes may be simplified.

The device 400 may include an active shape controller as described above. Accordingly, by activating the shape controller, the form or profile of the device may be changed to a desired shape or form. Other embodiments may include passive shape controllers, solid material, a pre-formed shape, or other device features as described herein.

FIG. 10B shows leads 406 extending outwardly from the reaction activator, which may be connected to a power source as described above. In some embodiments, such as wireless embodiments, the reaction activator may not include leads extending outside the implantable device 400.

Some implantation processes may include curing or hardening the device 400 during the implantation procedure by activating the reaction activator. The hardening process may include monitoring temperatures using any of the features or process steps disclosed above, including monitoring temperatures with thermocouples and using timers and power variations to obtain a desired curing profile.

In some implantation processes, the hardening or curing occurs after the surgical site is closed. For example, the hardening or curing may occur later in the surgery, post-operative, at home, in an office visit, or at other times or places. Later curing may allow a patient to optimize the placement of the device by determining at what position the vertebrae are most comfortable. In these embodiments, the leads may extend from a surgical site in a manner similar to a drain tube. Later, perhaps during an office visit after surgery, the patient may align his or her vertebrae to a comfortable position by, for example, bending over until any pain is alleviated. In that position, the reaction activator can be powered by the leads to initiate hardening of the implantable device in a position that provides the most relief to the spinal joint. After curing or hardening is complete, when the patient stands erect, the affected spinal joint is maintained in the comfortable position because the affected vertebrae, such as at the spinous processes, are secured in position. Once hardened, the leads 406 may be removed from the reaction activator and from the implantable device or alternatively, the leads 406 and the reaction activator may be removed from the implantable device 400. These may be percutaneously removed through the skin or through a tube sheath. Alternatively, the leads and/or the reaction activator may be left in the patient or in the device 400. It should be noted that in some embodiments, only portions of the device, such as a wing 112a from FIG. 5 may be hardened in place, while in other embodiments, the entire device is hardened in place.

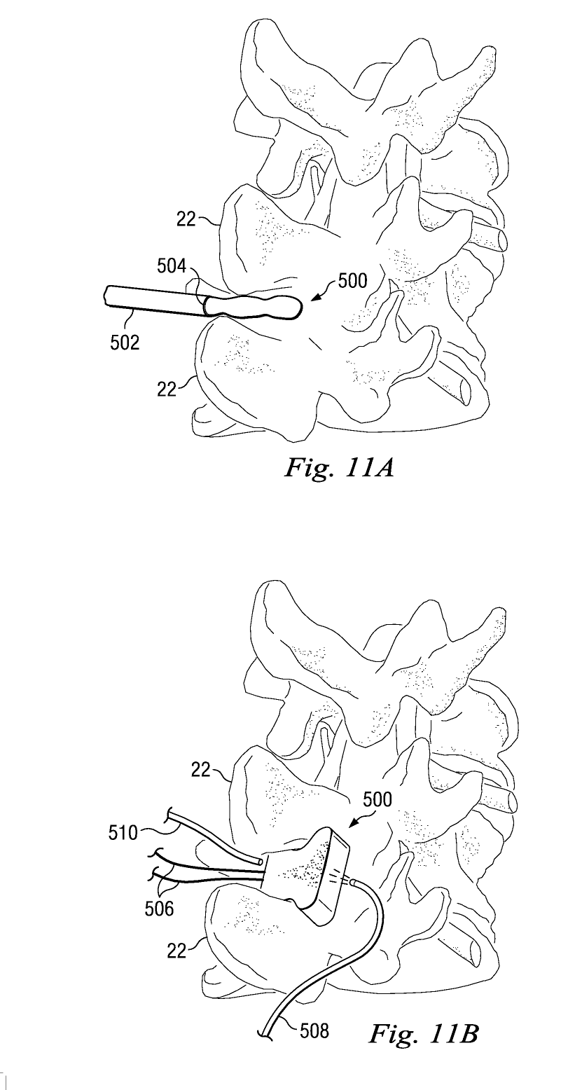

FIGS. 11A and 11B show an implantable device 500 being placed in an un-expanded or deflated state and expanded to support and stabilize upper and lower spinous processes 22 according to one embodiment of the present invention. The implantable device 500 may include any of the features of the embodiments described in this disclosure.

As an initial step, a rod (not shown) is inserted into the patient until its end is positioned at the application point. In this embodiment, the application point will be between the adjacent spinous processes 22. A conduit 502 is then slid over the rod until its end 504 is positioned at the point proximate to the rod end. The rod may than be removed leaving only the conduit 502 in the patient.

At this stage, referring now to FIG. 11A, the implantable device 500 is deployed from the conduit end 504 between the upper and lower spinous processes. In FIG. 11A, the implantable device 500 is in a deflated or unexpanded shape. In some embodiments, a reaction activator may be disposed within the implantable device 500. A hardenable material is pumped through the conduit 500 and into the device 500 to expand it as illustrated in FIG. 11B. The size and volume of the device 500 increases as the hardenable material enters. The physician may monitor the volume of material delivered and/or a pressure indicator.

Upon complete deployment, the conduit 504 may be removed from the implantable device 500 or alternatively, it may be left in place until the hardenable material is hardened in place. Any opening or port formed in the implantable device 500 may be sealed to completely enclose the hardenable material, or alternatively may remain unsealed as the open/exposed hardenable material cures to form a seal.

The shape of the implantable device 500 may be controlled, if desired, by the jacket having a preformed shape, or by passive or active shape controllers as described above. In the embodiment shown, the reaction activator is encompassed by the jacket of the device 500 and leads 506 extend outwardly from the reaction activator for connection to a power source. In some embodiments, such as the wireless embodiments, the reaction activator may not include leads extending outside the implantable device 500. As explained above, the implantation process may include curing or hardening the device 500 during or after the implantation procedure by activating the reaction activator.

In some implantation processes, the curing may be monitored using light sensors configured to monitor the curing process. Such a system is shown in FIG. 11B, where implanting the device 500 employs a light sensor including a light source 508 and a light detector 510. In these embodiments, the light source 508 is an external light configured to radiate on the implantable device 500 through an optical fiber. The light detector 510 may be disposed to detect the amount of light diffusing through the device 500 and may be on an opposite side of the device 500.

During curing, opacity of some polymers increases, diffusing the light. The light detector 510 may be connected to a photo-resistor that may monitor the amount of light diffusing through the device. As curing occurs, the light penetration changes and the change can be detected by the photo-resistor. The amount of detected light may be used to provide instantaneous or real-time feedback to the power source to control the hardening process, such as by increasing or decreasing the voltage (e.g., 5-30 volts) as the device 500 catalyzes or becomes cloudy or clearer. In some embodiments, instead of an external light source, the light source may be disposed within the device 500 in a manner similar to the reaction activator. In these embodiments, the light source may be, for example, a white light LED. Although the light source 508 and the light detector 510 are shown on opposing sides of the device 500, in some embodiments, they are on the same side and the detector monitors reflected light. Other systems also may be used.

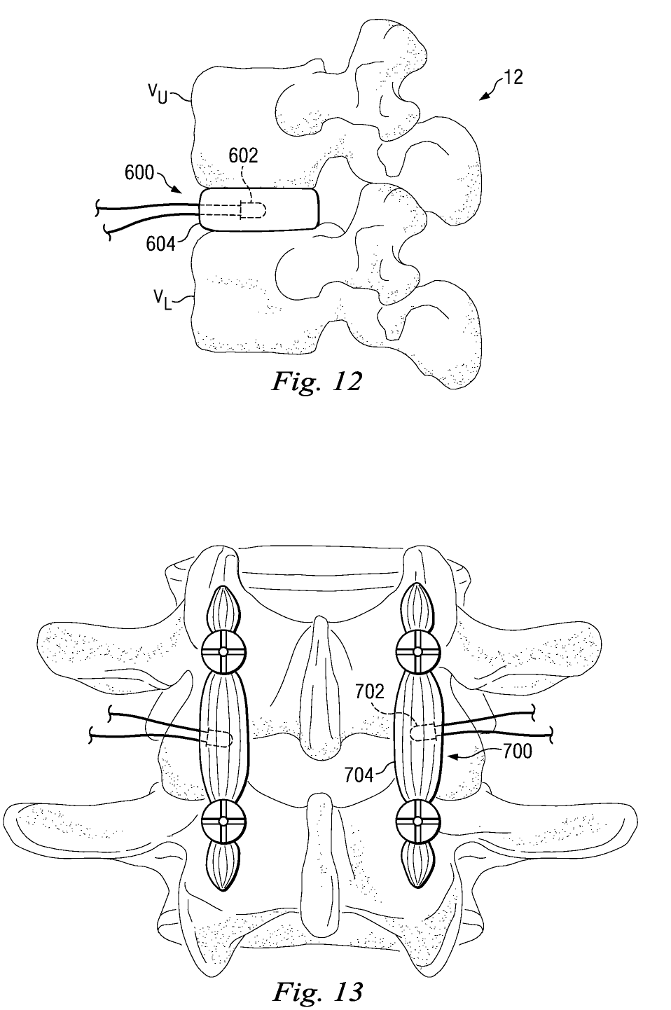

In some embodiments, the reaction activator is disposed outside, rather than being disposed within or being encompassed by the jacket of the implantable device. Also, in some embodiments, such as the embodiment shown in FIG. 12, the implantable device is an intervertebral nucleus replacement or augmentation device disposed between upper and lower vertebrae. The device 600 may include any of the features discussed above. FIG. 12 shows the device 600 having a reaction activator 602 disposed within a jacket 604. In other embodiments, such as the embodiment shown in FIG. 13, the implantable device is an injectable spinal rod configured for posterior placement on an upper and lower vertebrae. The device 700 may include any of the features discussed above. As shown, a reaction activator 702 may be disposed within a jacket 704. Other embodiments are contemplated. For example, in some embodiments, the implantable device is a flexible or moldable posterior instrumented spinal rod. In others, the implantable device is a flexible tube and tether arrangement.

In some implementations, the implantable device may be configured for more than one activation. For example, hardening the hardenable material with the reaction activator may occur during implantation or afterward, such as during an office visit after surgery. Later, additional adjustments to the implantable device may be made using the same or a different reaction activator. For example, an implantable device may include multiple reaction activators disposed in multiple regions. One of the reaction activators may initiate a reaction in one region to change the hardness or stiffness of the device in that region. Later, another reaction activator may initiate a reaction in another region to change the hardness or stiffness in that region, thereby incrementally changing the stiffness of the implantable device.

Changing the stiffness by hardening the material also may be done incrementally. For example, the reaction activator may be used to initiate the hardening process but not fully harden the hardenable material. Later, if additional support becomes desirable, the reaction activator may be reactivated to initiate additional hardening to change the stiffness of the implantable device.

In yet other embodiments, the active shape controller is incrementally activated to change stiffness or device shape post-surgically. For example, the shape controllers may be activated once during implantation and activated yet again during a later office visit to affect the height, the shape, or other features of the implantable device.

Such incremental treatment may allow physicians to monitor the patient and determine post-surgically the desired stiffness for the implantable device. Because not all patients require the same levels of support or stiffness, this post-operative customizing may relieve strain at the vertebrae and may address possible causes of post-operative pain.

Access to the surgical site may be through any surgical approach that will allow adequate visualization and/or manipulation of the bone structures. Example surgical approaches include, but are not limited to, any one or combination of anterior, antero-lateral, posterior, postero-lateral, transforaminal, and/or far lateral approaches. Implant insertion can occur through a single pathway or through multiple pathways, or through multiple pathways to multiple levels of the spinal column. Minimally invasive techniques employing instruments and implants are also contemplated.

It is understood that all spatial references, such as “top,” “inner,” “outer,” “bottom,” “left,” “right,” “anterior,” “posterior,” “superior,” “inferior,” “medial,” “lateral,” “upper,” and “lower” are for illustrative purposes only and can be varied within the scope of the disclosure. Also, cure and harden are terms used interchangeably throughout this disclosure to describe a hardening material. These terms are meant to encompass any material that hardens over time, and are not limited to curing materials. Further, we note that any of the features of one of the embodiments of the implantable devices may be combined with any of the features on any of the others and that because this description does not discuss every conceivable combination of features is not a limitation on the description or the scope of the application. For example only, any embodiment may include one or more than one reaction activator and any embodiment may employ thermocouples, and any embodiment may include an access port, etc. Also, while embodiments of the invention may be applied to the lumbar spinal region, embodiments also may be applied to the cervical or thoracic spine or between other bone structures.

While embodiments of the invention have been illustrated and described in detail in the disclosure, the disclosure is to be considered as illustrative and not restrictive in character. All changes and modifications that come within the spirit of the invention are to be considered within the scope of the disclosure.

We claim:

1. A vertebral stabilizing device for stabilizing adjacent vertebrae, comprising:
a jacket formed of a biocompatible material and being configured for implantation between the vertebrae, the jacket being configured to encompass a hardenable material; and
a reaction activator encompassed by the jacket.

2. The vertebral stabilizing device of claim 1, further comprising:
a hardenable material encompassed by the jacket.

3. The vertebral stabilizing device of claim 1, wherein the reaction activator is an ultraviolet LED.

4. The vertebral stabilizing device of claim 1, wherein the reaction activator is one of an infrared LED and a generator.

5. The vertebral stabilizing device of claim 1, including a plurality of reaction activators.

6. The vertebral stabilizing device of claim 1, comprising regions, the reaction activator being configured to activate the hardenable material within a specific region.

7. The vertebral stabilizing device of claim 1, comprising a shape controller.

8. The vertebral stabilizing device of claim 7, wherein the shape controller is a passive shape controller.

9. The vertebral stabilizing device of claim 7, wherein the shape controller is an active shape controller.

10. The vertebral stabilizing device of claim 7, wherein the shape controller is part of the jacket.

11. The vertebral stabilizing device of claim 1, wherein the jacket comprises recesses configured to receive spinous processes.

12. A system for stabilizing adjacent vertebrae, comprising:
a vertebral stabilizing device including
a jacket formed of a biocompatible material and being configured for implantation between the vertebrae, the jacket being configured to encompass a hardenable material, and
a reaction activator encompassed by the jacket; and
a power source configured to power the reaction activator.

13. The system of claim 12, further comprising a light detector configured to monitor the amount of light from the vertebral stabilizing device.

14. The system of claim 12, wherein the power source is a wireless power source.

15. The system of claim 12, wherein the vertebral stabilizing device include leads and wherein the power source is attached to the leads.

16. The system of claim 12, comprising a thermocouple configured to detect temperatures of the vertebral stabilizing device.

17. The system of claim 12, wherein the power source is disposed outside a patient's body.

18. The system of claim 12, wherein the reaction activator is configured to initiate a reaction to harden a hardenable material in only a portion of the device.

19. A system for posterior stabilization of vertebrae, comprising:
a jacket formed of a biocompatible material and being configured for implantation between a first spinous process of an upper first vertebra and a second spinous process of a lower second vertebra to provide posterior support to the first and second vertebrae,
a hardenable material disposed within the jacket; and
a reaction activator operable to initiate a reaction of the hardenable material to increase the hardness of the hardenable material.

20. The system for posterior stabilization of vertebrae of claim 19, wherein the reaction activator is encompassed by the jacket.

22. The system for posterior stabilization of vertebrae of claim 20, wherein the reaction activator is an ultraviolet light emitting diode.

23. A method of stabilizing adjacent vertebrae, comprising:
implanting a jacket formed of a biocompatible material between an upper and a lower vertebrae, the jacket being configured to encompass a hardenable material; and
activating a reaction activator encompassed by the jacket to harden a hardenable material encompassed by the jacket.

24. The method of claim 23, including percutaniously removing leads associated with the reaction activator from the reaction activator.

25. The method of claim 23, including removing the leads and the reaction activator from the jacket.

26. The method of claim 23, including monitoring the hardening of the hardenable material using one of an external light sensor and a thermocouple.

27. The method of claim 23, including changing the shape of the jacket after implanting the jacket by applying voltage to piezoelectric materials.

28. The method of claim 23, wherein activating the reaction activator includes providing power to the reaction activator with a wireless energy source.

29. The method of claim 23, wherein activating the reaction activator includes powering the reaction activator with an external energy source.

30. The method of claim 23, including closing a surgical site providing access to the vertebrae prior to activating the reaction activator.

31. The method of claim 23, including activating the reaction activator hardens the hardenable material in only a portion of the device.

32. The method of claim 23, wherein the jacket encompasses the hardenable material prior to the implantation step.

33. The method of claim 23, wherein the jacket encompasses the reaction activator prior to the implantation step.

34. The method of claim 23, including activating the reaction activator more than one time to incrementally affect the stiffness of the implantable device.

35. A method of stabilizing a posterior portion of vertebrae, comprising:
implanting a jacket formed of a biocompatible material between a first spinous process of an upper first vertebra and a second spinous process of a lower second vertebra to provide posterior support to the first and second vertebrae, the jacket being configured to encompass a hardenable material;
exposing the hardenable material to a reaction activator source that initiates a reaction of the hardenable material to increase the hardness of the hardenable material.

36. The method of stabilizing of claim 34, wherein the reaction activator is encompassed by the jacket.
